(19) United States
(12) Patent Application Publication
(10) Pub. No.: US 2026/0157686 A1
(43) Pub. Date: Jun. 11, 2026

(54) METHOD AND SYSTEM FOR CLASSIFYING AND MITIGATING THE SEVERITY LEVEL OF DYSARTHRIA

(71) Applicant: Chung Yuan Christian University, Taoyuan City (TW)

(72) Inventor: Fu-Yu CHEN, Taoyuan City (TW)

(21) Appl. No.: 18/975,588

(22) Filed: Dec. 10, 2024

(51) Int. Cl. A61B 5/00; G10L 15/06; G10L 15/16

(52) U.S. Cl. CPC A61B 5/4803; A61B 5/4082; A61B 5/7203; A61B 5/7264; G10L 15/063; G10L 15/16; A61B 2505/09; A61B 2560/0247

(57) ABSTRACT

Disclosed herein is a system for classifying the severity level of dysarthria in a subject having Parkinson's Disease (PD). The system includes a language training module for producing a plurality of acoustic recordings of the PD subject, an artificial intelligence (AI)-based module for classifying the severity level of dysarthria based on speech features extracted from the plurality of acoustic recordings. Also provided herein is a method for mitigating the severity of dysarthria of a PD subject. The method includes determining the severity level of dysarthria of the PD subject by using the present system; suggesting one or more language training exercises to the PD subject; and instructing the PD subject to practice the suggested one or more language training exercises to mitigate the severity level of dysarthria.

### BACKGROUND OF THE INVENTION

### 1. Field of the Invention

The present disclosure relates to a system and a method for classifying the severity level of dysarthria. More particularly, the disclosure invention relates to a system and a method for improving dysarthria that provides training to a person with dysarthria based on the classified result.

### 2. Description of Related Art

Importance of early diagnosis and mitigation for speech disorder (e.g., dysarthria) has been increased. The dysarthria is a motor speech disorder most commonly found in subjects with Parkinson's diseases (PD). It results from impaired movement of the muscle used for speech production. Traditional assessment on severity level of dysarthria is not only time-consuming but also often suffers from a lack of intra-rater reliability due to their subjective nature. This variability in assessment significantly affects the choice and effectiveness of treatment strategies for dysarthria. To achieve optimal treatment outcome, personalized treatment is crucial, which depends on correct early diagnosis.

Accordingly, there exists in the related art a need of an improved method and/or system for classifying dysarthria subjects effectively and efficiently according to their severity levels in speech, so that proper treatments may be allocated to mitigate the symptoms associated with dysarthria.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In one aspect, the present disclosure is directed to a system for classifying the severity level of dysarthria in a Parkinson's Disease (PD) subject. The system comprises:
a language training module for producing a plurality of acoustic recordings of the PD subject who is instructed to perform the following tasks (i) and (ii) for one or more times, in which each of the plurality of acoustic recordings is directed to either task (i) or task (ii):
   (i) pronouncing a vowel sound for 5-12 seconds; and
   (ii) reading a script of short sentences;
an artificial intelligence (AI)-based module comprising a first, a second, and a third Support Vector Machine (SVM) classifiers for classifying the severity level of dysarthria based on speech features extracted from the plurality of acoustic recordings as “normal”, “mild”, “moderate” or “severe”;
   wherein,
the speech features are used as data

$${\{ ( {x}_{i} , {y}_{i} ) \}}_{i = 1}^{N}$$

for the AI-based module, where xᵢ is a feature vector and its associated label is yᵢ∈{0,1,2,3}, for each binary problem y>k, its positively labeled training dataset

$${X}_{k}^{+}$$

and negatively labeled training dataset

$${X}_{k}^{-}$$

are constructed as Equation (1):

$${X}_{k}^{+} = \{ ( {x}_{i} , 1 ) \vert {y}_{i} > k \} , {X}_{k}^{-} = \{ ( {x}_{i} , - 1 ) \vert {y}_{i} \leq k \} , \qquad ( 1 )$$

$$k = 0 , 1 , 2$$

each SVM k aims to obtain a hyperplane that maximizes the margin between the classes y>k and y≤k.

According to embodiments of the present disclosure, each speech feature belongs to any one of phonation, articulation, prosody, or spectral feature categories.

According to embodiments of the present disclosure, the first SVM classifier is trained to distinguish the severity level of “normal” (y≤0) from “mild”, “moderate” and “severe” (y>0); the second SVM classifier could distinguish the severity level of “normal” and “mild” (y≤1) from “moderate” and “severe” (y>1); and the third SVM classifier could distinguish the severity level of “normal”, “mild” and “moderate” (y≤2) from “severe” (y>2). The three SVM classifiers function in an integrated manner to collectively differentiate the severity levels of dysarthria in individuals with Parkinson's disease.

According to optional embodiments of the present disclosure, the system further comprises a pre-language training module programmed to perform the following tasks (1) to (2):
(1) determining ambient noise of the PD subject; and
(2) determining the distance between a recorder and the PD subject;
wherein,
when the determined ambient noise in task (1) exceeds 50 decibels (dbs), the pre-language training module will instruct the PD subject to relocate to another location where the ambient noise is below 50 dbs; and
when the determined distance in task (2) is above or under 35 cm, the pre-language training module will instruct the PD subject to move closer or away from recorder.

In another aspect, the present disclosure is directed to a method for classifying the severity level of dysarthria in a Parkinson's Disease (PD) subject via use of the present system. The method comprises:
(a) invoking the language training module to instruct the PD subject to perform the following tasks (i) and (ii) for one or more times while recording each performance thereby producing a plurality of acoustic recordings with each acoustic recording being directed to either task (i) or task (ii),
   (i) pronouncing a vowel sound for 5-12 seconds; and
   (ii) reading a script of short sentences;
(b) transmitting the plurality of acoustic recordings of step (a) to the AI-based module to extract speech features therefrom; and
(c) classifying the severity level of dysarthria based on the extracted speech features of step (b) by using the first, second and third SVM classifiers of the AI-based module.

According to embodiments of the present disclosure, in step (a), at least 20 acoustic recordings of the PD subject are produced.

According to embodiments of the present disclosure, each speech feature belongs to any one of phonation, articulation, prosody, or spectral feature categories.

According to embodiments of the present disclosure, the first SVM classifier is trained to distinguish the severity level of “normal” (y≤0) from “mild”, “moderate” and “severe” (y>0); the second SVM classifier could distinguish the severity level of “normal” and “mild” (y≤1) from “moderate” and “severe” (y>1); and the third SVM classifier could distinguish the severity level of “normal”, “mild” and “moderate” (y≤2) from “severe” (y>2). The three SVM classifiers function in an integrated manner to collectively differentiate the severity levels of dysarthria in individuals with Parkinson's disease.

In a further aspect, the present disclosure aims to provide a method for mitigating the severity level of dysarthria of a PD subject. The method comprises:
(a) determining the severity level of dysarthria of the PD subject by using the present method;
(b) suggesting one or more language training exercises to the PD subject; and
(c) instructing the PD subject to practice the suggested one or more language training exercises of step (c) to mitigate the severity level of dysarthria.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a diagram depicting a system 100 for classifying the severity level of dysarthria in a PD subject 10 according to one exemplary embodiment of the present disclosure;

FIG. 2 depicts a flow chart of a method 200 for improving the severity level of dysarthria of a PD subject according to one exemplary embodiment of the present disclosure;

FIG. 3A depicts a flow chart of a method for implementing S210 of the method 200 of FIG. 2;

FIG. 3B depicts a flow chart of a method for implementing S220 of the method 200 of FIG. 2; and

FIG. 4 depicts the confusion matrix of the predictions for the proposed model under LOSO setup in accordance with Example 2 of the present disclosure.

### DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

According to exemplary embodiments of the present disclosure, a system, method and computer product for classifying the severity level of dysarthria in a Parkinson's Disease (PD) subject, suggesting one or more language training exercises to the PD subject, and/or assisting the PD subject to practice the suggested language training exercises are provided.

### 1. An Overview of the Present System

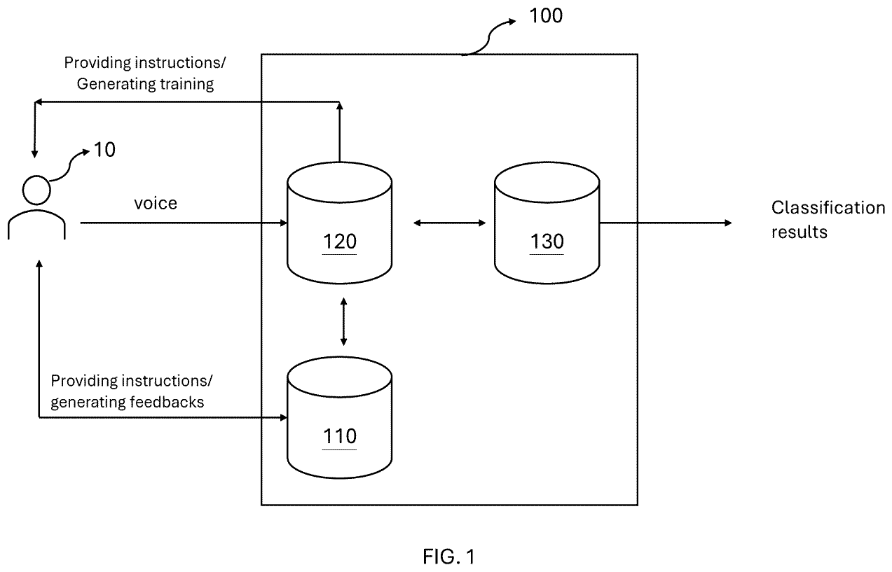

FIG. 1 is a schematic diagram depicting a system 100 for classifying the severity level of dysarthria in a PD subject 10 according to an exemplary embodiment of the present disclosure. The system 100 comprises a language training module 120, an artificial intelligence (AI)-based module 130, and optionally, a pre-language training module 110, operably coupled to each other. In general, the language training module 120 is programmed to instruct the user 10, who is a PD subject with dysarthria, to perform certain language tasks (e.g., reading a paragraph), the performance is recorded simultaneously as acoustic recordings and are transmitted to the AI-based module 130 for classification and further analysis.

According to embodiments of the present disclosure, the user 10 is instructed to perform language tasks one or more times, and each performance is recorded simultaneously thereby producing one or more recordings. According to embodiments of the present disclosure, the language tasks may include, (i) pronouncing a vowel sound (e.g., the sound of “ae”) for 5-12 seconds; and (ii) reading a script that consists of multiple short sentences. Note that the short sentences may be adapted from well-known examples used by speech-language pathologists (SLPs) for the diagnosis of severity level of dysarthria. According to embodiments of the present disclosure, at least 20 recordings are produced, with each recording being directed to either task (i) or task (ii). According to preferred embodiments of the present disclosure, each recording is a soundtrack that consists only acoustic data. Alternatively, or optionally, the recording may be a video that includes both image and acoustic data. Preferably, the present disclosure employs only acoustic data for subsequent analysis and classification.

According to some embodiments of the present disclosure, the acoustic data collected from PD subjects with the severity levels of dysarthria already being classified by SLPs is used as speech data to train a machine learning model (e.g., Support Vector Machine (SVM) classifiers) thereby establishing the present AI-based module 130, which in turn, is used to classify speech data collected from un-classified PD subject with dysarthria and provide feedback to the language training module 120. The establishment of the AI-based module 130, and the classification of severity level of dysarthria by the AI-based module 130 will be described in detail in later part of the present disclosure.

Optionally, or in addition to classification, the AI-based module 130 may also provide feedback to the language training module 120, which then generates a list of proposed language training exercises based on the feedback (i.e., the PD subject's classification result), so that the user 10 may exercise to mitigate or improve his/her level of dysarthria or delay the progression of dysarthria.

Optionally, or in addition, the system 100 may further include an optional pre-language training module 110, which is programmed to ensure each language task performed by the user 10 takes place in a controlled environment. Preferably, prior to the performance of language tasks, the pre-language training module 110 gives instruction to the user 10 (i.e., the PD subject) to perform following tasks (1) and (2):
(1) determining ambient noise of the user 10; and
(2) determining the distance between a recorder and the user 10;

According to embodiments of the present disclosure, when the determined ambient noise in task (1) exceeds 50 decibels (dbs), the pre-language training module 110 will instruct the user 10 to relocate to a quieter place where the ambient noise is below 50 dbs; and when the determined distance in task (2) is above or under 35 cm, the pre-language training module 110 will instruct the user 10 to move closer or away from the recorder.

Examples of the recorder suitable for use in the present disclosure include, but are not limited to, a sound recorder, a video recorder, a smartphone, and the like. According to preferred embodiments of the present disclosure, a smartphone is used to record the tasks performed by the user 10. According to embodiments of the present disclosure,

According to embodiments of the present disclosure, the pre-language training module 110, the language training module 120 and the AI-based module 130 are stored in the memory of a server, which the user 10 may access remotely through an application (APP) from the recorder (e.g., a smartphone of the user 10). Alternatively, or optionally, the language training module 120 and the AI-based module 130 are stored in the memory of a server, while the pre-language training module 110 is stored in the APP of the recorder, which may be the smartphone of the user 10.

### 2. Establishing the AI-Based Module

To establish the present AI-based module 130, clinical data (i.e., the acoustic data collected from PD subjects with classified severity levels of dysarthria) is used as speech data to train a machine learning model, preferably, Support Vector Machine (SVM) classifiers.

According to embodiments of the present disclosure, each PD subject is first instructed to perform language tasks set forth by the language training module 120 thereby producing at least 20 acoustic recordings as described above in the Overview Section of this paper. The acoustic recordings collected from each PD subject are then reviewed and classified by SLPs based on Grade, Roughness, Breathiness, Asthenia, Strain (GRBAS) scale, which served as the basis for categorizing each patient into groups of severity: “Normal,” “Mild,” “Moderate,” and “Severe.” Speech features are then extracted from the acoustic recordings of these classified PD subjects by exploring well-known speech libraries to capture common dysarthria symptoms across different speech dimensions. According to preferred embodiments of the present disclosure, a total of 182 speech features independently belong to phonation, articulation, prosody, or spectral feature categories are extracted from the speech libraries, which are further narrowed down to a concise list of 23, specifically associated with dysarthric speech disorders.

According to embodiments of the present disclosure, the AI-based module 130 comprises a first, a second and a third SVM classifiers, each classifier is trained by the extracted speech features obtained from the classified PD subjects described above thereby establishing the present AI-based module 130. Specifically, the extracted speech features are used as training data

$${\{ ( {x}_{i} , {y}_{i} ) \}}_{i = 1}^{N}$$

to train the AI-based module 130, where xᵢ is a feature vector and its associated label is yᵢ∈{0,1,2,3}, for each binary problem y>k, its positively labeled training data

$${X}_{k}^{+}$$

and negatively labeled training dataset

$${X}_{k}^{-}$$

are constructed as Equation (1):

$${X}_{k}^{+} = \{ ( {x}_{i} , 1 ) \vert {y}_{i} > k \} , {X}_{k}^{-} = \{ ( {x}_{i} , - 1 ) \vert {y}_{i} \leq k \} , \qquad ( 1 )$$

$$k = 0 , 1 , 2$$

each SVM k aims to obtain a hyperplane that maximizes the margin between the classes y>k and y≤k.

According to embodiments of the present disclosure, the first SVM classifier could distinguish the severity level of “normal” (y≤0) from “mild”, “moderate” and “severe” (y>0); the second SVM classifier could distinguish the severity level of “normal” and “mild” (y≤1) from “moderate” and “severe” (y>1); and the third SVM classifier could distinguish the severity level of “normal”, “mild” and “moderate” (y≤2) from “severe” (y>2).

### 3. Methods for Mitigating the Severity Level of Dysarthria of a PD Subject

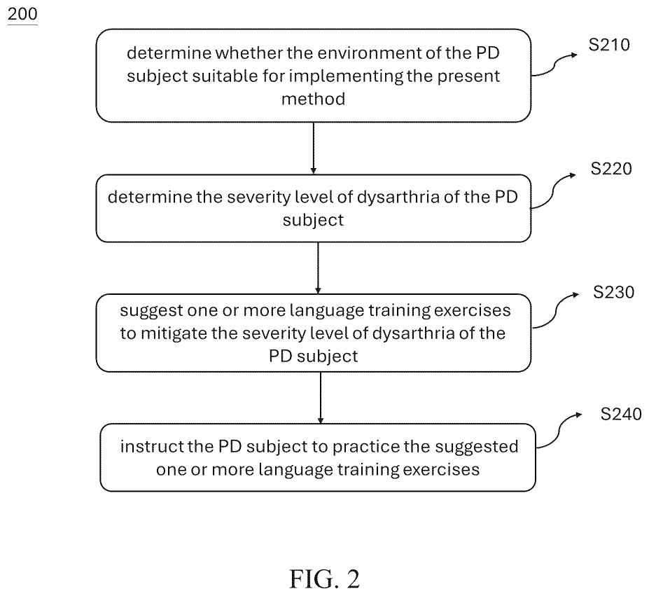

Also encompasses in the present disclosure is a method for mitigating the severity level of dysarthria of a PD subject with the aid of the present system. Reference is made to FIG. 2, which is a flowchart of a method 200 for mitigating the severity level of dysarthria of a PD subject with the aid of the present system 100. The method 200 comprises steps of:
S210: determine whether the environment of the PD subject suitable for implementing the present method;
S220: determine the severity level of dysarthria of the PD subject;
S230: suggest one or more language training exercises to mitigate the determined level of dysarthria of the PD subject; and
S240: instruct the PD subject to practice the suggested one or more language training exercises to mitigate his/her severity level of dysarthria.

In general, the method commences by a user (i.e., the PD subject) activates a recorder, such as his/her smartphone, which has an application stored therein, the application, when activated, will automatically execute the present method 200. Upon activation, the pre-language training module 110 of the system 100 will implement S210 of the method 200, in which the user is instructed to implement steps that ensure subsequent language tasks will take place in a controlled environment. Note that S210 is an optional step and may be omitted by the user if he/she deems the surrounding environment suitable for performing language tasks.

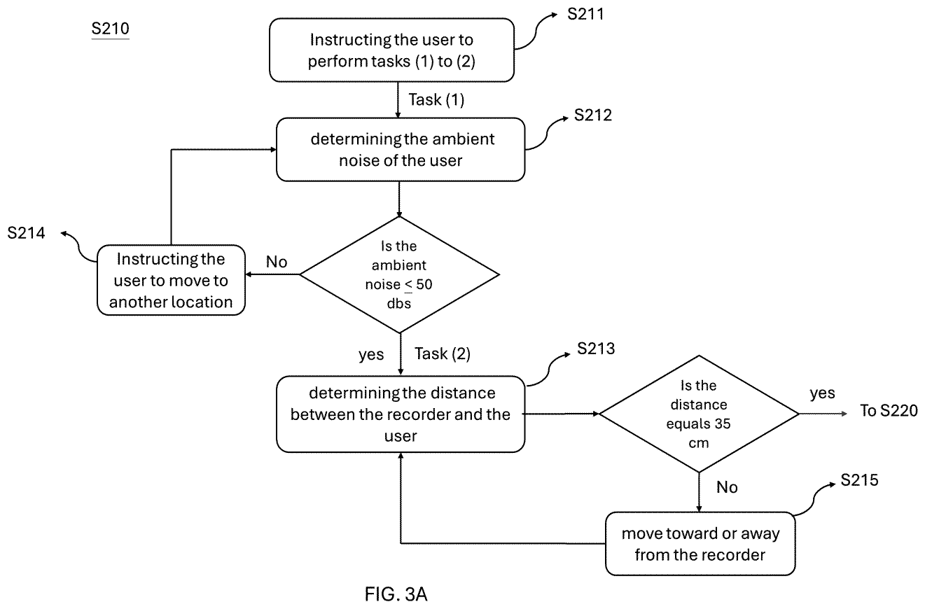

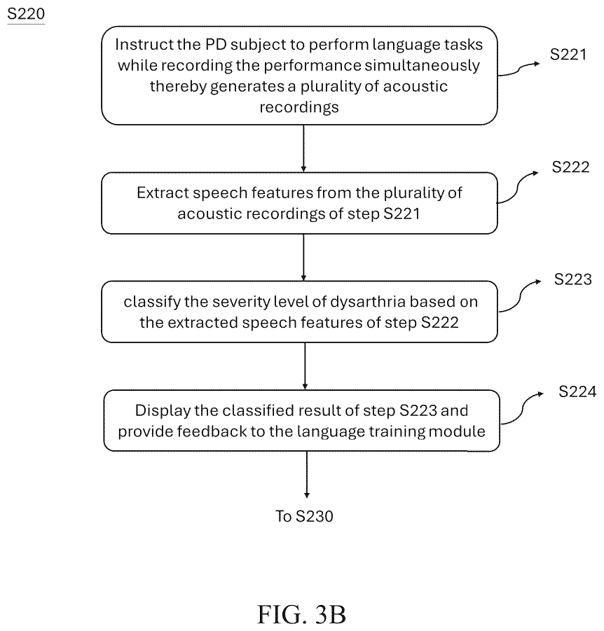

Step S210 may include further steps. Referring to FIG. 3A, in step S211, the pre-language training module 110 gives instruction to the user to perform tasks (1) to (2), in which task (1) is to determine the ambient noise of the user (S212); and task (2) is to determine the distance between the recorder and the user (S213). In S212, when the determined ambient noise exceeds 50 decibels (dbs), the pre-language training module 110 will instruct the user to relocate to another location (i.e., a quieter place) (S214), and repeat the ambient noise determination step S212 again until the ambient noise is below 50 dbs. In the case when the determined ambient noise in step S212 is lower than 50 dbs, then the process will go the next step of S213.

In S213, the pre-language training module 110 gives instruction to the user to perform the following 3 steps for task (2): (a) placing the recorder (e.g., a smartphone) on a table with the camera of the recorder being leveled with the eyes of the user, (b) measuring the distance between the camera and the eyes, and (c) calculating the distance between the recorder and the user via use of the measured distance of step (b). In the case when the determined distance in task (2) is above or under 35 cm, the pre-language training module 110 will instruct the user to move closer or away from the recorder (S215) and repeat the distance determination step S213 again until the distance is about 35 cm. In the case when the determined distance in step S213 is about 35 cm, then the method 200 will go the next step S220 of the method 200, in which both the language training module 120 and the AI-based module 130 of the present system 100 are invoked.

Step S220 may include further steps. Referring to FIG. 3B, in step S221, the language training module 120 is invoked to instruct the user to perform language tasks while each performance is recorded simultaneously. The language tasks include (i) pronouncing a vowel sound (e.g., the sound of “ae” and the like) for 5-12 seconds; and (ii) reading a script of short sentences, which may be adapted from well-known examples used by SLPs for the diagnosis of severity level of dysarthria. According to embodiments of the present disclosure, at least 20 recordings are produced by each user in S221, with each recording being directed to either task (i) or task (ii). According to preferred embodiments of the present disclosure, each recording is a soundtrack that consists only acoustic data. Alternatively, or optionally, the recording may be a video that includes both image and acoustic data. Preferably, the present disclosure employs only acoustic data for subsequent analysis and classification.

The plurality of acoustic recordings produced in S221 are then transmitted to the AI-based module 130 of the present system 100, in which speech features specifically associated with dysarthric speech disorders (e.g., any one of the features listed in Table 3 of Example 1.1) are extracted from the plurality of acoustic recordings (S222). The extracted speech features are then used as speech data for the AI-based module 130, in which the SVM classifiers therein classify the severity level of dysarthria of the PD subject (S223) as “normal”, “mild”, “moderate” or “severe” based on the received speech data.

The classified result of the PD subject in S223 may be displayed on the recorder in real time to inform the user about his/her current condition (S224). Optionally, or in addition, the result may also be stored in the present system 100 as part of the user's health record for further use and/or reference.

Optionally, or in addition, the AI-based module 130 may provide feedback to the language training module 120 (S224), which may then generate one or more language training exercises based on the classified result (S230). The language training module 120 may then have the PD subject practice the suggested language training exercises so as to mitigate the severity level of dysarthria of the PD subject (S240).

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLES

### Materials and Methods

### Datasets and Experimental Setup

This database was composed of 40 Mandarin-speaking subjects recruited from National Cheng Kung University Hospital (Tainan, Taiwan), consisting of 18 females and 22 males. The 34 patients with PD had an average age of 68.4±8.1 years (mean±SD) and a mean disease duration of 6.7±4.7 years. In accordance with the Hoehn and Yahr staging scale, all patients were in stages 1-4 (1-1.5 as early stage, 2-3 as middle stage, and 4 as late stage). Detailed patient description is shown in Table 1.

Recordings were conducted using a smartphone, which had a sampling rate of 44.1 KHz. When recording participant data, all sessions were conducted in quiet rooms, such as meeting rooms, consultation rooms, or the participant's home, where the ambient noise level was below 50 decibels. The phone was placed on a tabletop, recording at a distance of about 35 cm. The script for the participant to read was displayed on the phone screen during the recording. Following the recommendation of speech language pathologists (SLP), part of the Mandarin Hearing in Noise Test (MHINT) was chosen for the second version of the reading material. Each participant recorded 20 pieces of speech data, where each datum was 5 s in duration.

TABLE 1
Demographic and clinical details of subjects at the time of data collection

| | Normal | Mild | Moderate | Severe |
|---|---|---|---|---|
| GRBAS Score | 0 | 1-3 | 3-7 | >7 |
| Number of Subjects | 6 | 13 | 11 | 10 |
| Age (years) | 61.2 ± 7.1 | 65 ± 9.5 | 69.5 ± 5.4 | 70.8 ± 7.1 |
| Hoehn and Yahr (μ ± σ) | — | 2.3 ± 0.9 | 3.1 ± 0.8 | 2.7 ± 0.9 |
| Time since Diagnosis (μ ± σ) | — | 5.2 ± 3 | 7.1 ± 4.2 | 8.3 ± 6.2 |
| GRBAS Score (μ ± σ) | — | 1.9 ± 0.7 | 4.8 ± 1.1 | 8.5 ± 1.3 |

The GRBAS scale was used to evaluate the patient's language abilities. It is a unified scoring system primarily used in speech pathology and research for assessing audio quality. It has been shown to correlate well with acoustic parameters. Three medical experts were invited to score the patients' recorded files by using this scale. The scores were then averaged to derive the final GRBAS scores (G, R, B, A, and S) for each patient, which served as the basis for categorizing them into groups of severity: “Normal” (score=0), “Mild” (0<total score≤3), “Moderate” (3<total score <=7), and “Severe” (total score >7).

### Speech Feature Extraction and Selection

Several speech analysis libraries, including python_speech_features, disvoice, parselmouth, and openSMILE eGeMAPSv02 were explored in this study. The python_speech_features library provides common speech features for automatic speech recognition (ASR), such as MFCCs and filterbank energies. Disvoice computes features related to glottal activity, phonation, articulation, prosody, and phonological aspects. Parselmouth extracts various acoustic parameters, including duration, mean and standard deviation of fundamental frequency (F0), HNR, jitter, shimmer, and spectral features. OpenSMILE eGeMAPSv02 offers frequency-related parameters (e.g., pitch, jitter, and formant frequencies), energy/amplitude-related parameters (e.g., shimmer, loudness, and HNR), spectral parameters (e.g., alpha ratio, Hammarberg index, spectral slopes, and MFCCs), and temporal features (e.g., rate of loudness peaks, lengths of voiced and unvoiced regions, and pseudo syllable rate). A substantial collection of speech disorder features was obtained in the feature extraction phase. However, handling such an extensive array of features carried challenges, predominantly due to the curse of dimensionality. An excess of features can increase model complexity, lead to overfitting, and decrease model interpretability. For these reasons, forward feature selection was used to select the most impactful subset of features to optimize the predictive capacity of the proposed model. This method started with an empty set and then incrementally incorporated features that most significantly improve the model's performance, halting when the addition of further features no longer provided a substantial enhancement. Meanwhile, 20% of the data was set aside as a validation set for the feature selection process. During each iteration, the features that significantly enhanced accuracy based on validation set were identified. Any new feature that met this criterion was added to the curated set of features, serving as the foundation for the next iteration. This selection process was distinct for each classifier, necessitating repetition for each one.

Ordinal Ranking Framework with SVM for PD Dysarthria Severity Classification

Ordinal ranking methods are more appropriate because they capture the ordered nature of the severity levels while handling their nonuniform intervals. This ordinal ranking strategy aimed to enhance the accuracy of ML and DL models in the severity classification for PD dysarthria. Considering that the database had 40 patients only, this study opted for a simpler model, specifically choosing SVM with a linear kernel, to mitigate the risk of overfitting. The following ordinal ranking approach could also be trained using any classification models.

The weighted binary classification method proposed by Lin and Li was adopted to solve this ordinal ranking problem (Lins and Li, Advance in Neural Information Processing Systems, 2006, 19). In this approach, a weight was assigned to each category, and the cost of prediction errors was correlated with these weights. This method allows the use of a simpler binary classification algorithm to handle the ordinal ranking problem as the “larger than label y” ordering property. Specifically, the four levels of PD dysarthria severity were labeled as y∈{0,1,2,3} (where 0 is normal, 1 is mild, 2 is moderate, and 3 is severe). These binary “larger than” classification problems were solved separately in a cost-sensitive learning framework, allowing flexible binary classifiers with distinctive features to be constructed to best fit different sub-problems. In the ordinal ranking with SVM framework, three SVMs were trained to separate the labels into groups: SVM1 to separate normal (y≤0) from mild, moderate and severe (y>0); SVM2 to separate normal and mild (y≤1) from moderate and severe (y>1); and SVM3 to separate normal, mild, and moderate (y≤2) from severe (y>2).

Given the training data

$${\{ ( {x}_{i} , {y}_{i} ) \}}_{i = 1}^{N} ,$$

where xᵢ is a feature vector and its associated label is

$${X}_{k}^{+}$$

yᵢ∈{0,1,2,3}, for each binary problem y>k, its positively labeled training dataset and negatively labeled training dataset

$${X}_{k}^{-}$$

are constructed as follows:

$${X}_{k}^{+} = \{ ( {x}_{i} , 1 ) \vert {y}_{i} > k \} , {X}_{k}^{-} = \{ ( {x}_{i} , - 1 ) \vert {y}_{i} \leq k \} , \qquad ( 1 )$$

$$k = 0 , 1 , 2$$

Each SVM k aimed to obtain the hyperplane that maximizes the margin between the classes y>k and y≤k. The first SVM aimed to distinguish category 0 (Normal) from categories 1-3 (Mild, Moderate, and Severe). If the actual value exceeded 0 in this classifier, the label value was set to 1; otherwise, −1 was used. The second SVM aimed to distinguish between categories 0 and 1 (Normal and Mild) and categories 2 and 3 (Moderate and Severe). For this classifier, the label value was set to 1 when the actual value was greater than 1; otherwise, it was set to −1. Similarly, the third SVM aimed to differentiate between categories 0-2 (Normal, Mild, and Moderate) and category 3 (Severe). The optimization problem for each SVM can be formulated as follows:

$${min}_{{w}_{k} , {ξ}_{ik ,} {b}_{k}} \frac{1}{2} {\vert \vert {w}_{k} \vert \vert}^{2} + {\sum }_{i = 1}^{N} {c}_{i k} {ξ}_{i k} \qquad ( 2 )$$

$$subject to$$

$$( 2 [ {y}_{i} > k ] - 1 ) ( {{w}_{k}}^{T} {x}_{i} + {b}_{k} ) \geq 1 - {ξ}_{ik} f or i = 1 … N \qquad ( 3 )$$

$${ξ}_{i k} \geq 0 ,$$

where wₖ and bₖ are the weight vector and bias term k-th SVM, ξᵢₖ are the slack variables, and cᵢₖ is the cost for misclassifying the i-th sample in the k-th SVM. The cost was designed as follows: cᵢₖ=C|2yᵢ−(2k+1)|, where C is the regularization parameter as in standard SVM. Such design makes sure that the cost is higher when yᵢ is farther from k, which punishes serious error like classifying a severe sample to normal.

During the prediction phase, the final prediction was determined by sequentially using the trained SVM models. The first SVM model was utilized for an initial prediction. If the predicted value exceeded 0 (i.e., the model predicted the “Mild,” “Moderate,” or “Severe” category), the final prediction result was incremented by 1. This process was followed by a second prediction using the second SVM model, where the final prediction result was incremented by 1 if the predicted value exceeded 0 (i.e., the model predicted the “Moderate” or “Severe” category). In the case of the third SVM model, if the predicted value exceeded 0, the final prediction result was further incremented by 1 (indicating a “Severe” prediction). The prediction outcome was ultimately determined on the basis of the final accumulated value: “Normal” was assigned if the value was 0, “Mild” if the value was 1, “Moderate” if the value was 2, and “Severe” if the value was 3.

Initialize: Set the initial prediction result p = 0.
SVM1: Predict using ŷ₁ = sign(w₁ · x + b₁). if ŷ₁ > 0, increment p by 1.
SVM2: Predict using ŷ₂ = sign(w₂ · x + b₂). if ŷ₂ > 0, increment p by 1.
SVM3: Predict using ŷ₂ = sign(w₃ · x + b₃). if ŷ₃ > 0, increment p by 1.
Final Prediction: The final prediction result p determines the severity level:
p = 0: Normal, p = 1: Mild, p = 2: Moderate, p = 3: Severe

### Other Classification Algorithms

As mentioned above, a SVM classifier with ordinal ranking was used in this study. Previous studies have explored various ML models and DL approaches for dysarthria severity classification. In this study, four classification approaches were implemented for performance comparison: SVM multiclass; SVR; and DL methods, including DNN and LLM with LoRA. The performance of SVM with ordinal ranking was compared to that of the DNN DL approach within the ordinal ranking framework to identify the most effective method for accurately classifying dysarthria severity levels. LOSO cross-validation was used, leaving the sample of one individual out for validation, to ensure the model learns disease-specific features. Forward feature selection was implemented to extract the most suitable features for each classifier algorithm.

Performance metrics provide quantifiable measures that describe how well the model is performing on given data. For this study, the performance metrics used were accuracy and the square root of the mean squared error (RMSE), both calculated for in-sample and out-of-sample data. These metrics served distinct purposes and provided a holistic view of the model's effectiveness. For training the models, each sentence was treated as a data point. For evaluation, the prediction for a subject was considered as a whole by averaging the predictions of all sentences from that patient and rounding it to the nearest whole number. Other evaluation metrics include Precision, Recall, F1-score, and confusion matrix.

DNN with Ordinal Ranking

The classical DNN model was implemented in PyTorch by stacking three dense layers with ReLU activation functions and a dropout factor of 0.4. Each DNN was trained with a batch size of 8 and a learning rate of 1e-4, over 100 epochs. The model parameters included 32 neurons per hidden layer, a total of three layers, activation functions of ReLU and Sigmoid, and batch normalization. The Adam optimizer, known for its computational efficiency and minimal memory requirements, was utilized. The training method aligned with the previously mentioned SVM ordinal ranking approach to perform ordinal ranking. For LOSO cross-validation, an early stopping approach was implemented to halt training sessions before overfitting occurred.

### ML-Based Multiclass Classification Approaches

SVM is a supervised ML algorithm typically used for classification tasks, aiming to find an optimal boundary between the possible outputs. In this experiment, a linear kernel with an optimal regularization parameter C=0.05 was used. For multiclass SVM classification tasks, a one-against-all approach was chosen to break down multiclass problems into multiple binary classification problems. The SVM multiclass model had an input dimension of 10 and four classes.

Support Vector Regression (SVR) was employed to predict continuous numeric values such as severity scores, using the same hyperparameters as the multiclass SVM. The SVR model utilized the GRBAS score as the ground truth, normalized from 0 to 3 to align with the multiclass targets (classes 0-3).

### DL-Based Classifiers

A DNN model was implemented with a regression layer as the final layer. The hyperparameters of this model were consistent with those used in DNN with ordinal ranking approach, including 32 neurons per hidden layer, a total of three layers, ReLU activation functions, and no dropout or batch normalization. However, the output layer consisted of a single node, and Mean Square Error (MSE) was utilized to compute the loss. Similar to the support vector regression model, the GRBAS scores were normalized from 0 to 3 as ground truth, and forward feature selection was performed. Each DNN was trained with a batch size of 8 and a learning rate of 1e-4 for 100 epochs.

Whisper is a pretrained model for ASR tasks, trained on a large dataset of diverse audio (680,000 h). In this experiment, the encoder was extracted from this encoder-decoder architecture and a classification head was added for severity score classification tasks. One of the most popular parameter-efficient fine-tuning methods, LoRA of Large Language Models, which involves adding a smaller number of new weights to the model and training these weights only, was applied to reduce the amount of expensive computing power and labeled data required. This approach results in a quicker and less memory-intensive training process. A LoRA rank of 16 and an alpha of 32 were chosen because they are commonly recommended. Given that training LoRA models is time consuming, instead of using the LOSO method, one-third of the subjects were allocated as test data and the remaining subjects were used for training to evaluate the model's performance. For feature selection, each raw sentence was transformed into a log Mel-spectrogram and padded to a fixed length with zero, resulting in features shaped as (mel=80, sequence_length=3000) being fed into the model. During the training process, 50 warm-up steps were applied to stabilize training. The model was trained with a batch size of 8 and a learning rate of 1e-4 for 100 epochs.

### Example 1: Establishing Artificial Intelligence (AI)-Based Support Vector Machine with Ordinal Ranking (SVM-Ordinal) Learning Model

To build the present AI-based SVM-ordinal learning model, 40 PD subjects were recruited and respectively asked to perform multiple language tasks thereby producing audio data that were used to train and establish the system. The language task included: (1) pronouncing a vowel sound (e.g., “a”) for 5-12 seconds; and (2) reading a script of short sentences several times. An audio recording was made for each task performed, and a total of 20 audio recordings were obtained from each PD subject according to the procedures described in “Material and Methods” Section.

All the collected raw audio recordings were then filtered for feature extraction in Example 1.1 before being passed to an artificial neural network (i.e., a linear SVM classifier) for speech severity classification evaluation in Example 1.2 thereby establishing the present SVM-ordinal Learning model, which exhibited a final accuracy of 72% for sentence and 75% for person.

### 1.1 Feature Extraction

### Analysis of Selected Speech Features

In the initial stage of speech analysis, a broad array of 182 speech-related features was extracted from the well-known speech analysis libraries mentioned in “Material and Methods” Section. Table 2 presents a description of commonly used feature groups in previous literature, including phonation, articulation, prosody, and spectral features, along with the corresponding number of variants within each group. Through the forward feature selection process as described in “Material and Methods” Section, the initial set of features was refined to a concise list of 23, specifically associated with dysarthric speech disorders. The finalized set of features, along with their respective IDs, are cataloged in Table 3.

TABLE 2
Description of the extracted speech features

| | | Total |
|---|---|---|
| Feature Category | Description | Features |
| Phonation | Mean and standard deviation of fundamental frequency | 58 |
| | derivatives, jitter, shimmer, amplitude perturbation, | |
| | pitch perturbation, log energy, HNR, local jitter and | |
| | shimmer, perturbation quotients, and jitter and | |
| | shimmer features from different analyses | |
| Articulation | Frequencies, bandwidths, and amplitudes of formants | 18 |
| | F1, F2, and F3, including their mean and standard | |
| | deviation for different analyses | |
| Prosody | Mean, standard deviation, percentiles, and slopes of | 30 |
| | F0; loudness and related features; and measures of | |
| | voiced/unvoiced segments and equivalent sound level | |
| Spectral | MFCCs, spectral flux, formant frequencies, | 76 |
| | bandwidths, and amplitudes, including their mean and | |
| | standard deviation for different analyses | |

TABLE 3
Description of the selected feature used in the proposed method

| Classifier | Feature ID | Features |
|---|---|---|
| Classifier 1 | 4 | MFCC-average |
| | 8 | MFCC-average |
| | 13 | MFCC-std |
| | 28 | MFCC-average |
| | 104 | loudness_sma3_amean |
| | 40 | MFCC-average |
| | 49 | MFCC-std |
| | 2 | MFCC-skew |
| | 108 | loudness_sma3_percentile80.0 |
| Classifier 2 | 4 | MFCC average over time |
| | 8 | MFCC average over time |
| | 48 | MFCC average over time |
| | 128 | HNRdBACF_sma3nz_amean |
| | 168 | mfcc4V_sma3nz_amean |
| | 138 | F1amplitudeLogRelF0_sma3nz_amean |
| | 89 | localdbShimmer |
| | 91 | aqpq5Shimmer |
| | 160 | spectralFluxV_sma3nz_amean |
| Classifier 3 | 4 | MFCC average over time |
| | 12 | MFCC average over time |
| | 32 | MFCC average over time |
| | 36 | MFCC average over time |
| | 118 | mfcc2_sma3_amean |
| | 142 | F2bandwidth_sma3nz_amean |
| | 41 | MFCC std over time |
| | 137 | F1bandwidth_sma3nz_stddevNorm |
| | 22 | MFCC skewness over time |

### 1.2 Speech Severity Classification Evaluation

By employing the refined feature set, the linear SVM classifiers were trained with the above weighted binary classification setup of Example 1.1. As the dataset was collected from 40 subjects, Leave-one-subject-out (LOSO) cross-validation was used to train and evaluate the proposed model. At each iteration, the voice samples from one subject were used as a validation set, and the remaining samples were used as a training set to train and evaluate the model. This process was repeated for each person to calculate the validation accuracy. The cost parameter C was selected in the SVM-ordinal learning model that gave the best validation accuracy, and the final accuracy was 72% for sentence and 75% for person.

### Example 2: Comparison of the Severity Prediction of PD Subjects Obtained from Various Machine Learning Models

Various machine learning models including the SVM-ordinal Learning model established in Example 1.2, DNN-ordinal, SVM-MC, SVR, LLM-MC, and DNN were then used to evaluate severity level of PD subjects.

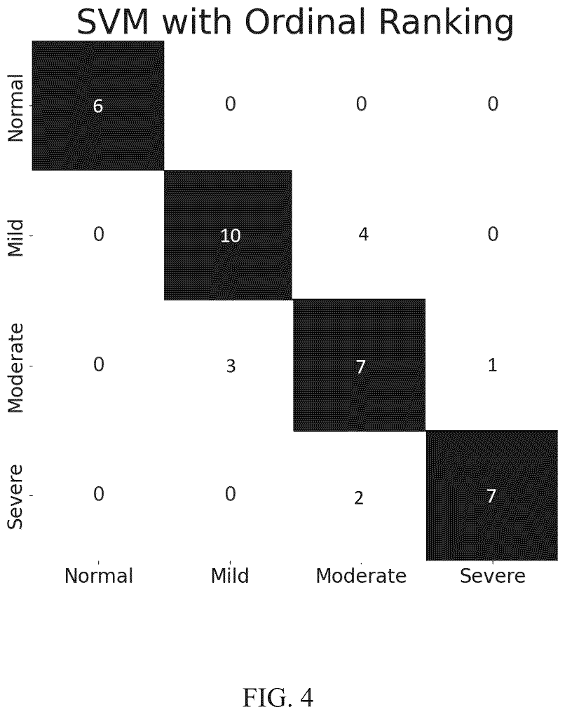

Table 4 shows the prediction for patients in each severity level. Considering that 75% accuracy was reached in the SVM-ordinal learning model of Example 1.2, the ordinal ranking approach has an additional advantage over simple multiclass classification. The classifier can precisely predict whether a person is healthy (class 0) or not. Furthermore, as shown in FIG. 4, for people with dysarthria, the prediction was at most one severity level away from the actual severity level. These properties are a result of optimizing for squared error, which places more penalty when the prediction is further from the actual severity level.

TABLE 4
Experiment results of different ML models
as categorized by severity level

| Model | Severity Level | Precision | Recall | F1-score |
|---|---|---|---|---|
| SVM-ORDINAL | Normal | 1 | 1 | 1 |
| | Mild | 0.71 | 0.77 | 0.74 |
| | Moderate | 0.64 | 0.54 | 0.58 |
| | Severe | 0.78 | 0.87 | 0.82 |
| DNN-ORDINAL | Normal | 1 | 0.83 | 0.91 |
| | Mild | 0.67 | 0.71 | 0.69 |
| | Moderate | 0.5 | 0.64 | 0.56 |
| | Severe | 0.83 | 0.56 | 0.67 |
| SVM-MC | Normal | 1 | 0.67 | 0.8 |
| | Mild | 0.69 | 0.79 | 0.73 |
| | Moderate | 0.45 | 0.45 | 0.53 |
| | Severe | 0.78 | 0.78 | 0.78 |
| SVR | Normal | 0.64 | 0.54 | 0.58 |
| | Mild | 0.42 | 0.53 | 0.46 |
| | Moderate | 0.67 | 0.45 | 0.53 |
| | Severe | 1 | 1 | 1 |
| LLM-MC | Normal | 0.69 | 0.69 | 0.69 |
| | Mild | 0.6 | 0.8 | 0.69 |
| | Moderate | 0.86 | 0.5 | 0.63 |
| | Severe | 0 | 0 | 0 |
| DNN | Normal | 0.69 | 0.69 | 0.69 |
| | Mild | 0.6 | 0.8 | 0.69 |
| | Moderate | 0.86 | 0.5 | 0.63 |
| | Severe | 0 | 0 | 0 |

The comparison of ML and DL approaches by using ordinal ranking for PD dysarthria severity classification yielded significant insights. The SVM with ordinal ranking (i.e., SVM-ordinal) outperformed other models, achieving perfect precision, recall, and F1-score of 1 for the “Normal” category. It also demonstrated strong performance across “Mild,” “Moderate,” and “Severe” categories, with an overall balanced F1-score. The SVM multiclass model showed high precision for the “Normal” category (1) but had lower performance for other severity levels, particularly “Moderate,” where the recall and F1-score were 0.45 only. SVR had an overall lower performance, especially for the “Mild” category, with an F1-score of 0.46, although it performed perfectly for the “Severe” category. The LoRA model exhibited good performance for “Moderate” severity but failed to classify “Severe” cases. The DNN model showed high performance for “Moderate” severity but struggled with “Severe” cases, yielding an F1-score of 0. The DNN with Ordinal Ranking provided a balanced approach, achieving high precision, recall, and F1-score for the “Normal” category and showing reasonable performance across other severity levels.

TABLE 5
Experiment results of different ML models under
LOSO setup (hold-out validation was used for LLM)

| Model | RMSE | Accuracy |
|---|---|---|
| SVM-ORDINAL | 0.613 | 0.75 |
| SVM-MC | 0.833 | 0.68 |
| SVR | 0.67 | 0.55 |
| LLM-MC | 0.67 | 0.67 |
| DNN | 0.66 | 0.66 |
| DNN-ORDINAL | 0.7 | 0.68 |

Table 5 shows the results of the dysarthria severity classification models, summarized in terms of LOSO RMSE and accuracy. The best results are highlighted in boldface. The SVM-ordinal learning model achieved the best performance, with a LOSO RMSE of 0.613 and an accuracy of 0.75, indicating its effectiveness in capturing the ordinal nature of dysarthria severity levels.

Taken together, the results clearly demonstrated that the present SVM-ordinal learning model allows for a refined classification that discriminates healthy individuals and those with PD dysarthria, with 100% accuracy, and categorizes the level of severity with a remarkable 75% accuracy in LOSO cross-validation.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A system for classifying the severity level of dysarthria in a Parkinson's Disease (PD) subject comprising:
a language training module for producing a plurality of acoustic recordings of the PD subject who is instructed to perform the following tasks (i) and (ii) for one or more times, in which each of the plurality of acoustic recordings is directed to either task (i) or task (ii):
(i) pronouncing a vowel sound for 5-12 seconds; and
(ii) reading a script of short sentences;
an artificial intelligence (AI)-based module comprising a first, a second, and a third Support Vector Machine (SVM) classifiers for classifying the severity level of dysarthria based on speech features extracted from the plurality of acoustic recordings as “normal”, “mild”, “moderate” or “severe”;
wherein,
the speech features are used as data
$${\{ ( {x}_{i} , {y}_{i} ) \}}_{i = 1}^{N}$$
for the AI-based module, where xᵢ is a feature vector and its associated label is yᵢ∈{0,1,2,3}, for each binary problem y>k, its positively labeled training dataset
$${X}_{k}^{+}$$
and negatively labeled training dataset
$${X}_{k}^{-}$$
are constructed as Equation (1):
$${X}_{k}^{+} = \{ ( {x}_{i} , 1 ) \vert {y}_{i} > k \} , {X}_{k}^{-} = \{ ( {x}_{i} , - 1 ) \vert {y}_{i} \leq k \} , \qquad ( 1 )$$
$$k = 0 , 1 , 2$$
each SVM k aims to obtain a hyperplane that maximizes the margin between the classes y>k and y≤k.

2. The system of claim 1, wherein each speech feature belongs to any one of phonation, articulation, prosody, or spectral feature categories.

3. The system of claim 2, wherein
the first SVM classifier is trained to distinguish the severity level of “normal” (y≤0) from “mild”, “moderate” and “severe” (y>0);
the second SVM classifier could distinguish the severity level of “normal” and “mild” (y≤1) from “moderate” and “severe” (y>1);
the third SVM classifier could distinguish the severity level of “normal”, “mild” and “moderate” (y≤2) from “severe” (y>2); and
the first, second and third SVM classifiers function in an integrated manner to collectively differentiate the severity levels of dysarthria in the PD subject.

4. The system of claim 3, further comprising a pre-language training module programmed to perform the following tasks (1) to (3):
(1) determining ambient noise of the PD subject;
(2) determining the distance between two eyes of the PD subject; and
(3) determining the distance between a recorder and the PD subject, wherein the recorder has a camera embedded therein and is placed in a manner that the camera is leveled with the two eyes of the PD subject;
wherein,
when the detected ambient noise exceeds 50 decibels (dbs), the pre-language training module will instruct the PD subject to relocate to another location where the ambient noise is below 50 dbs; and
when the distance between the recorder and the PD subject is above or under 35 cm, the pre-language training module will instruct the PD subject to move closer or away from recorder.

5. The system of claim 4, wherein
the recorder is a sound recorder, a video recorder or a smartphone; and
the recorder is programmed to implement instructions of the language training module, the pre-language training module or both.

6. A method for classifying the severity level of dysarthria in a Parkinson's Disease (PD) subject via use of the system of claim 1, the method comprises:
(a) invoking the language training module to instruct the PD subject to perform the following tasks (i) and (ii) for one or more times while recording each performance thereby producing a plurality of acoustic recordings with each acoustic recording being directed to either task (i) or task (ii),
(i) pronouncing a vowel sound for 5-12 seconds; and
(ii) reading a script of short sentences;
(b) transmitting the plurality of acoustic recordings of step (a) to the AI-based module to extract speech features therefrom; and
(c) classifying the severity level of dysarthria based on the extracted speech features of step (b) by using the first, second and third SVM classifiers of the AI-based module.

7. The method of claim 6, wherein in step (a), at least 20 acoustic recordings of the PD subject are produced.

8. The method of claim 6, wherein each speech feature belongs to any one of phonation, articulation, prosody, or spectral feature categories.

9. The method of claim 8, wherein
the first SVM classifier is trained to distinguish the severity level of “normal” (y≤0) from “mild”, “moderate” and “severe” (y>0);
the second SVM classifier could distinguish the severity level of “normal” and “mild” (y≤1) from “moderate” and “severe” (y>1);
the third SVM classifier could distinguish the severity level of “normal”, “mild” and “moderate” (y≤2) from “severe” (y>2); and
the first, second and third SVM classifiers function in an integrated manner to collectively differentiate the severity levels of dysarthria in the PD subject.

10. The method of claim 6, further comprising invoking a pre-language training module to perform the following tasks (1) to (3):
(1) determining ambient noise of the PD subject;
(2) determining the distance between two eyes of the PD subject; and
(3) determining the distance between a recorder and the PD subject, wherein the recorder has a camera embedded therein and is placed in a manner that the camera is leveled with the two eyes of the PD subject;
wherein,
when the detected ambient noise exceeds 50 decibels (dbs), the pre-language training module will instruct the PD subject to relocate to another location where the ambient noise is below 50 dbs; and
when the distance between the recorder and the PD subject is above or under 35 cm, the pre-language training module will instruct the PD subject to move closer or away from recorder.

11. The method of claim 10, wherein the recorder is a sound recorder, a video recorder or a smartphone; and the recorder is programmed to implement instructions of the language training module, the pre-language training module or both.

12. A method for mitigating the severity of dysarthria of a PD subject comprising:
(a) determining the severity level of dysarthria of the PD subject by using the method of claim 7;
(b) suggesting one or more language training exercises to the PD subject; and
(c) instructing the PD subject to practice the suggested one or more language training exercises of step (b) to mitigate the severity level of dysarthria.

13. The method of claim 12, further comprising, prior to step (a), steps of:
(1) determining ambient noise of the PD subject;
(2) determining the distance between two eyes of the PD subject; and
(3) determining the distance between a recorder and the PD subject, wherein the recorder has a camera embedded therein and is placed in a manner that the camera is leveled with the two eyes of the PD subject;
wherein,
when the detected ambient noise exceeds 50 decibels (dbs), the pre-language training module will instruct the PD subject to relocate to another location where the ambient noise is below 50 dbs; and
when the distance between the recorder and the PD subject is above or under 35 cm, the pre-language training module will instruct the PD subject to move closer or away from recorder.

14. The method of claim 13, wherein the recorder is a sound recorder, a video recorder or a smartphone; and the recorder is programmed to implement instructions of the language training module, the pre-language training module or both.
